(19) United States
(12) Patent Application Publication
(10) Pub. No.: US 2021/0262963 A1
(43) Pub. Date: Aug. 26, 2021

(54) GRAPHENE-BASED CHEMICAL SENSING DEVICE AND SYSTEM

(71) Applicant: Culvert Engineering Solutions, LLC, Campbell, CA (US)

(72) Inventors: Prasad Panchalan, San Jose, CA (US); Alberto Vidal, San Jose, CA (US); Sanjiv Bhatt, San Jose, CA (US)

(21) Appl. No.: 17/182,013

(22) Filed: Feb. 22, 2021

Related U.S. Application Data
(60) Provisional application No. 62/979,834, filed on Feb. 21, 2020.
(60) Provisional application No. 63/014,428, filed on Apr. 23, 2020.

(51) Int. Cl. G01N 27/12; G16C 20/70; G16C 20/10; G06N 3/08

(52) U.S. Cl. CPC G01N 27/125; G06N 3/08; G16C 20/10; G16C 20/70

(57) ABSTRACT

In certain embodiments, chemical sensing may be facilitated. In some embodiments, a fluid sample may be received at a sensing device having one or more chemical sensitivities. A reaction of the sensing device to a chemical in the fluid sample may be detected based on the one or more chemical sensitivities of the sensing device. For example, a sensing unit within the sensing device having a particular chemical sensitivity may react with a chemical in the fluid sample. In some embodiments, the reaction may be a change in resistivity or piezoresistivity. One or more chemicals in the fluid sample associated with the reaction of the sensing device may be identified. In some embodiments, machine learning models or neural networks may facilitate the identification of chemicals associated with the reaction.

### PRIORITY CLAIM

The present application claims priority to U.S. Provisional Patent Application No. 62/979,834 filed on Feb. 21, 2020 entitled “GRAPHENE-BASED CHEMICAL SENSING DEVICE AND SYSTEM” and U.S. Provisional Patent Application No. 63/014,428 filed on Apr. 23, 2020 entitled “GRAPHENE-BASED CHEMICAL SENSING DEVICE AND SYSTEM USING PIEZORESISTIVITY AND RESISTIVITY,” the contents of which are herein incorporated by reference in their entirety.

### FIELD OF THE INVENTION

The invention relates to chemical sensing using a graphene-based sensing device and system to detect chemicals in gaseous and liquid environments.

### BACKGROUND OF THE INVENTION

Advances in sensor, computing and software technologies have made it possible for computers to detect and identify smells or chemicals in the environment. However, these technologies are limited in their sensitivities and applications. For example, current technologies lack functionality for customizing types, amounts, combinations, and ratios of chemicals to be detected in gaseous and fluid samples. Additionally, current technologies lack sensitivity to detect low levels of chemicals, which are nonetheless harmful, in fluid or gaseous samples.

Current chemical sensing technologies are further limited in their selectivity, repeatability, and reliability. For example, graphene-based sensing systems may be highly sensitive but may have selectivity problems, as they may exhibit similar responses to different types of gases. This drawback may lead to false detecting of various chemicals. Non-repeatability is another drawback, as preparation of sensing materials, construction of gas sensors, building of experimental platforms, and characterization of parameters all contribute to the non-repeatability of current chemical sensing devices. Problems with reliability stem from degradation of manufactured sensors over time.

Many existing sensors have been demonstrated to have high sensitivity but poor repeatability and reliability. For example, metal oxide semiconductor sensors have high operating temperatures and high power consumption and are sensitive to sulfur poisoning. Metal oxide semiconductor field effect transistor sensors exhibit baseline drift and require a controlled environment. calorimetric sensors have high operating temperatures and risk of catalyst poisoning. Optical sensors have complex circuitry and low portability and suffer from photobleaching. Quartz crystal microbalance sensors have complex circuitry and are sensitive to humidity and temperature. Surface acoustic wave sensors have complex circuitry and are sensitive to humidity and temperature. Carbon nanofiber based sensors are expensive and difficult to fabricate and lack precision. Conducting polymer sensors are sensitive to humidity and temperature and may suffer from baseline drift and saturation. Carbon particle based sensors are sensitive to humidity and temperature and may suffer from baseline drift. Due to the drawbacks of current sensing systems, as described above, a sensing system that is selective, repeatable, and reliable is needed. These and other drawbacks exist.

### SUMMARY OF THE INVENTION

Aspects of the invention relate to methods, apparatuses, or systems for graphene-based sensing of chemicals and smells in various environments.

Some aspects include a permanent, replaceable, or single-use sensing cartridge that includes: a series of sensing units, wherein each of the sensing unit comprises: a base layer or carrier; a first layer based on graphene or graphene film functionalized with metal oxide (MOX) or DNA molecules via an intermediate functional group; a series of spacers between the sensing units of the series of sensing units; and a housing. Sensing units are stacked to form a single cartridge unit with a unique set of chemical selectivity that targets specific applications.

Some other aspects include a computer system for measuring the chemical reaction of a sample on a cartridge: a computer system that comprises one or more processors programmed with computer program instructions that, when executed, cause the computer system to: receive, at a sensing device having one or more chemical sensitivities, a fluid or gas sample; detect, based on the one or more chemical sensitivities of the sensing device, a reaction of the sensing device to a chemical in the sample; and identify the chemical in the sample associated with the reaction of the sensing device.

Some other aspects include a computer system for matching chemical reactions of a sample in a cartridge to a library or model of other chemical reactions: a computer system that comprises one or more processors where some of these processors are dedicated machine learning processors that accelerate chemical sample matching using locally stored machine learning models.

Some other aspects include a remote machine learning computer system for matching chemical reactions of a sample in a cartridge to a library or model of other chemical reactions: a wireless or wired communications system that sends sample measurements to the remote computer system, and receives processed results and outcomes.

Some other aspects include a sensitive, selective, repeatable, and reliable sensing device. The sensing device is able to differentiate between similar molecules, produce the same results as other identical devices, and maintain its properties over time. A graphene-based sensing system which identifies chemicals by measuring changes in piezoresistivity and resistivity in response to an interaction with the chemicals may be used to achieve the aforementioned objectives.

Some other aspects include a sensing system which is able to quickly identify chemicals with low power consumption. The small size of the sensing system described herein (e.g., chip scale) may expand the applications for which the system may be used.

Some other aspects include a remote machine learning computer system for matching chemical reactions of a sample in a cartridge to a library or model of other chemical reactions: a wireless or wired communications system that sends sample measurements to the remote computer system, and receives processed results and outcomes.

Various other aspects, features, and advantages of the invention will be apparent through the detailed description of the invention and the drawings attached hereto. It is also to be understood that both the foregoing general description and the following detailed description are examples and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

FIG. 1 shows a system for facilitating chemical sensing, in accordance with one or more embodiments.

FIG. 2 shows a machine learning model configured to facilitate chemical sensing, in accordance with one or more embodiments.

FIG. 3 shows a sensing unit, in accordance with one or more embodiments.

FIG. 4 shows a stack of sensing units, in accordance with one or more embodiments.

FIG. 5 shows a sensing system, in accordance with one or more embodiments.

FIG. 6 shows graphene deposited on a silicon test chip on a pressure sensor, in accordance with one or more embodiments.

FIG. 7 shows a device fabrication process, in accordance with one or more embodiments.

FIG. 8 shows a sensing unit and a sensing device, in accordance with one or more embodiments.

FIG. 9 shows an exposed measurement structure, in accordance with one or more embodiments.

FIG. 10 shows simultaneous excitation of an isolated reference structure and an exposed measurement structure, in accordance with one or more embodiments.

FIG. 11 shows a stress function with an isolated reference structure and an exposed measurement structure, in accordance with one or more embodiments.

FIG. 12 shows a plane view of a sensor, in accordance with one or more embodiments.

FIG. 13 shows a flowchart of a method of facilitating chemical sensing, in accordance with one or more embodiments.

FIG. 14 shows a flowchart of a method of facilitating sensitive, repeatable, and reliable chemical sensing, in accordance with one or more embodiments.

### DETAILED DESCRIPTION OF THE INVENTION

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

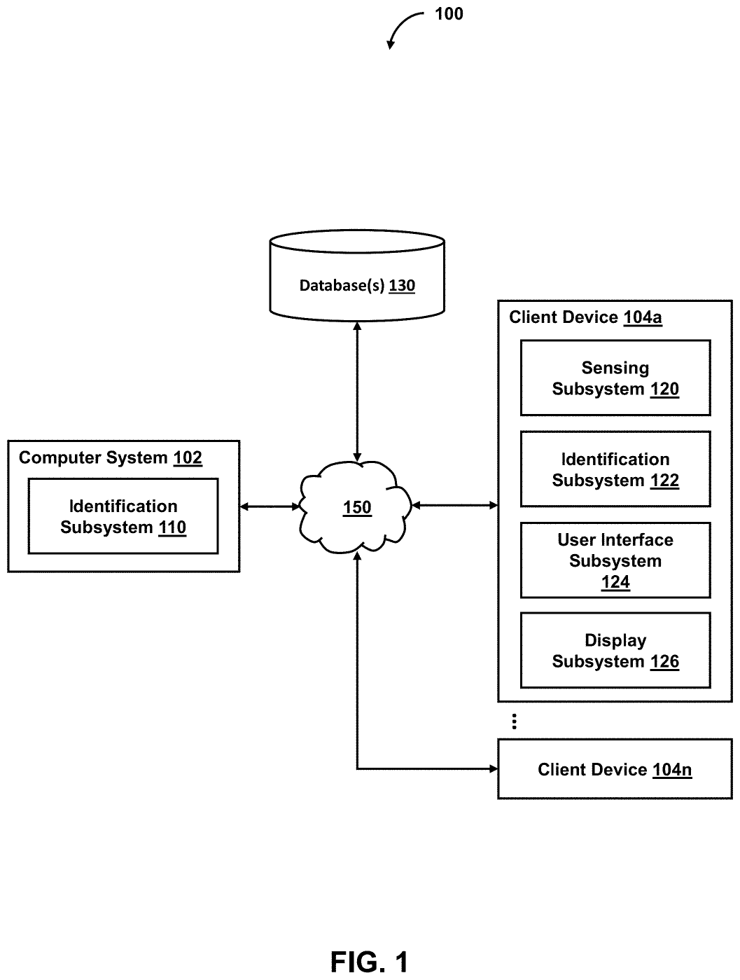

FIG. 1 shows a system 100 for facilitating chemical sensing, in accordance with one or more embodiments. As shown in FIG. 1, system 100 may include computer system 102, client device(s) 104 (or client devices 104a-104n), database(s) 130, or other components. Computer system 102 may include identification subsystem 110 or other components. Each client device 104 may include sensing subsystem 120, identification subsystem 122, user interface subsystem 124, display subsystem 126, or other components. Each client device 104 may include any type of mobile terminal, fixed terminal, or other device. By way of example, client device(s) 104 may include a desktop computer, a notebook computer, a tablet computer, a smartphone, a wearable device (e.g., augmented reality glasses or goggles), a handheld device, a device attachment, or another client device. Users may, for instance, utilize one or more client devices 104 to interact with one another, one or more servers, or other components of system 100. It should be noted that, while one or more operations are described herein as being performed by particular components of computer system 102, those operations may, in some embodiments, be performed by other components of computer system 102 or other components of system 100. As an example, while one or more operations are described herein as being performed by components of computer system 102, those operations may, in some embodiments, be performed by components of client device(s) 104.

In some embodiments, system 100 may facilitate chemical sensing and identification. System 100 may comprise a sensing device having one or more chemical sensitivities. For example, sensing devices may have one or more sensing units, each corresponding to one or more chemical sensitivities. The sensing units may have one or more coatings which provide the sensing units with one or more properties. In some embodiments, the sensing units may be combined to create a sensing device having particular chemical sensitivities. In some embodiments, the combination of the sensing units in the sensing device may correspond to a particular application for which the sensing device is to be used. In some embodiments, system 100 may receive a fluid sample at the sensing device. In some embodiments, the one or more chemical sensitivities may cause one or more sensing units of the sensing device to react to a chemical in the fluid sample. In some embodiments, system 100 may identify one or more chemicals associated with the reactions of the sensing units. For example, system 100 may compare the reactions to a database comprising reactions based on chemical sensitivities and chemicals associated with the reactions based on the chemical sensitivities. In some embodiments, system 100 may utilize a machine learning model or neural network in order to identify the chemicals based on the reaction of the sensing device. System 100 may therefore enable sensing and identification of chemicals present in fluids in a variety of environments, as discussed below in further detail.

In some embodiments, client device(s) 104 may comprise a sensing device. For example, a sensing device may comprise a combination of sensing units. In some embodiments, sensing units may comprise different types. For example, sensing units of a particular type may be manufactured together. In some embodiments, a series of sensing units (e.g., in stacks, sheets, molds, or other series) may be manufactured at one time. A series of sensing units may be treated with a variety of processes in order to provide the sensing units with various properties. In some embodiments, the stack of sensing units may be processed using heat, compression, layering, adhesion, or using any other processing techniques.

In some embodiments, sensing units may include a base layer. In some embodiments the base layer may be a sheet, cylinder, or other shape. In some embodiments, a base layer may be folded, wrapped, or otherwise manipulated to form a particular shape (e.g., a tube, prism, or other shape). In some embodiments, the base layer may be made of metal (e.g., stainless steel, copper, nickel, etc.), ceramic, or another material. In some embodiments, a series of sensing units may be coated with carbon or an allotrope of carbon, such as graphene. In some embodiments, graphene may be used due to its sensitive properties and ability to bond with chemicals (e.g., smells). In some embodiments, graphene may be applied as a layer onto the sensing units, inserted as a filler into the sensing units, placed within a cavity of the sensing units, or otherwise applied to the sensing units.

In some embodiments, the sensing units may additionally be coated with a chemical functionality dopant. For example, the dopant may be an impurity element which is added to the sensing unit in order to alter its properties. In some embodiments, the chemical functionality dopant may determine the type of sensing unit. For example, a particular chemical functionality dopant may be applied to a first series of sensing units, thereby adding a first chemical functionality to the first series of sensing units. The first series of sensing units may thereafter be a first type (e.g., type A) of sensing units. In some embodiments, different chemical functionality dopants may be applied to different series of sensing units such that multiple types of sensing units are manufactured.

In some embodiments, additional coatings or layers may be applied to the sensing units. For example, dielectric materials, which may insulate the sensing units from electric conduction, may be applied to the sensing units. Metal oxide, DNA dopants, or other layers may be applied to the sensing units to provide the sensing units with various properties. In some embodiments, each coating or layer may be applied using heat (e.g., in a furnace), with pipettes, or using other application techniques.

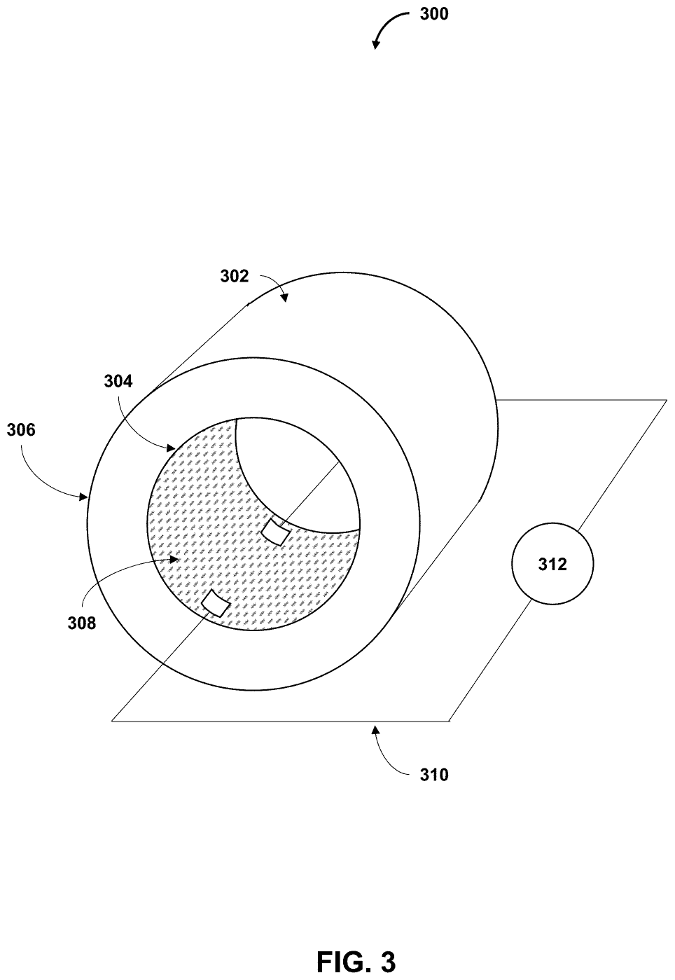

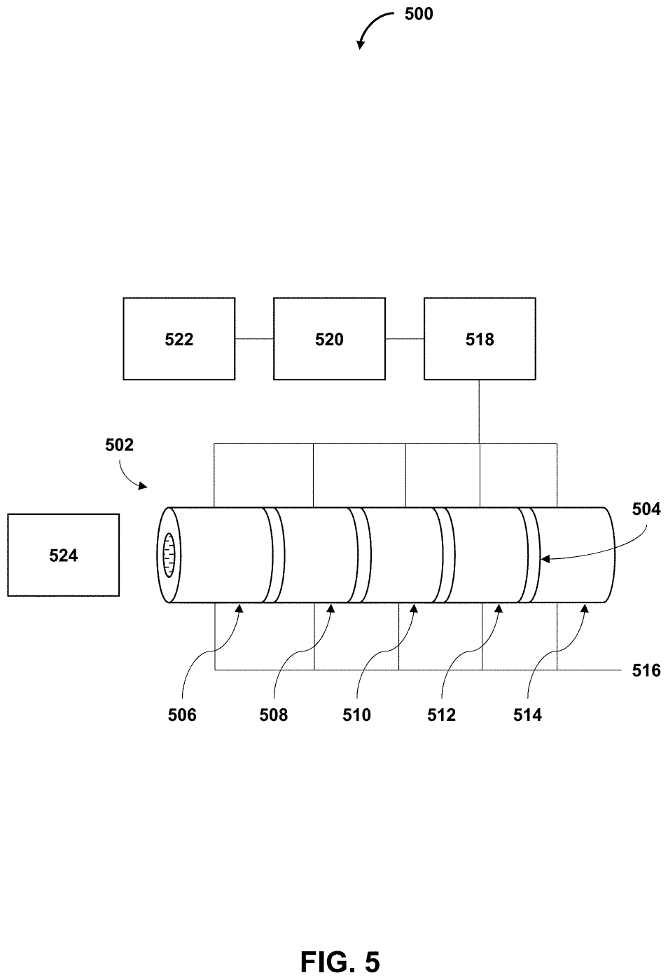

In some embodiments, a series of sensing units of a particular type (e.g., type A) may be broken apart after manufacturing. For example, if the series of sensing units is a column of stacked sensing units, the column may be broken apart into individual sensing units. In another example, if the series of sensing units is a sheet of sensing units, the sheet may be cut apart into individual sensing units. FIG. 3 shows a sensing unit 300, in accordance with one or more embodiments. As shown in FIG. 3, sensing unit 300 may comprise a base layer 302 (e.g., comprising stainless steel, ceramic, or some other material). In some embodiments, various materials may be attached to base layer 302, for example, on inside layer 304, on outside layer 306, in cavity 308, or in other applications. For example, graphene may be layered on inside layer 304 or may fill cavity 308. In some embodiments, chemical functionality dopants may be applied to inside layer 304. In some embodiments, chemical functionality dopants may be applied as chemical rinses, layers, textures (e.g., grooves), or other applications. In some embodiments, the particular chemical functionality dopants applied to sensing unit 300 may determine the type of sensing unit 300 (e.g., type A). In some embodiments, sensing unit 300 may be coated in a dielectric material. In some embodiments, metal oxides, DNA dopants, or other materials may be applied to sensing unit 300. For example, metal oxides or DNA dopants may be applied to inside layer 304, outside layer 306, cavity 308, or another portion of sensing unit 300. In some embodiments, metal oxides, DNA dopants, or other materials may function as catalysts or for other functions of sensing unit 300. In some embodiments, one or more coatings, layers, or other applied materials may fill cavity 308. In some embodiments, coatings, layers, or other applied materials may be porous such that fluid is able to pass through cavity 308. In some embodiments, an additional hole, channel, or cavity may be applied to sensing unit 300 such that fluids are able to pass through sensing unit 300. In some embodiments, connection 310 may connect sensing unit 300 to a device 312. In some embodiments, device 312 may be a voltage generator, an analog-to-digital converter (ADC), or some other device, as described in detail in relation to FIG. 5.

In some embodiments, sensing devices may be formed by combining sensing units of various types. In some embodiments, sensing units may be attached to form a sensing device using compression, adhesion, welding, interlocking, placement within a housing, or some other method for attaching the sensing units. In some embodiments, sensing units may be stacked, aligned, or otherwise combined to form a sensing device. In some embodiments, a first sensing device may be formed with a first combination of sensing units (e.g., types A, C, D, and E). In some embodiments, the first sensing device may have particular proportions of each type of sensing unit (e.g., two type A, one type C, 3 type D, 2 type E). In some embodiments, the particular combination and proportions of sensing units within a sensing device may be based upon an application for the sensing device. For example, a sensing device may be created to sense a particular chemical in a particular environment. In this example, the types and proportions of sensing units used to create the sensing device may be based upon the particular chemical or the particular environment.

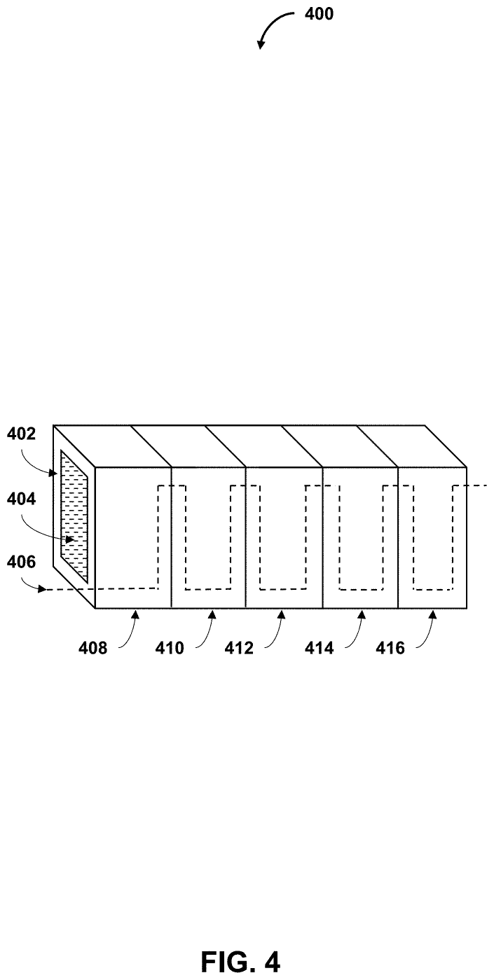

FIG. 4 shows a stack 400 of sensing units, in accordance with one or more embodiments. Sensing units, sensing stacks, or sensing devices may be formed in a variety of shapes, sizes, and configurations. In some embodiments, stack 400 may be comprised of blocks, sheets, cylinders, or other shaped sensing units. In some embodiments, stack 400 may be a sensing device or a part of a sensing device. In some embodiments, stack 400 may comprise a series of sensing units (e.g., sensing unit 408, sensing unit 410, sensing unit 412, sensing unit 414, sensing unit 416, etc.). In some embodiments, sensing units 408-416 may be attached to each other using compression, adhesion, welding, interlocking, placement within a housing, or some other method for attaching the sensing units. In some embodiments, the sensing units may comprise various types of sensing units. In some embodiments, sensing units 408-416 may be manufactured separately according to types of sensing unit. For example, sensing units 408-418 may be type A, type A, type B, type E, and type E, respectively. In another example, sensing units 408-416 may all be one type (e.g., type A) in order to amplify a signal associated with a particular chemical sensitivity. Sensing units of one type may be used when detecting a chemical that exists in low amounts in an environment. In some embodiments the types of sensing units may correspond to chemical functionality dopants that have been applied to the sensing units, as described above.

In some embodiments, sensing units 408-416 may each comprise chemical functionality dopants, graphene, metal oxides, DNA dopants, or other materials, on surfaces or in cavities of sensing units 408-416, as described above. In some embodiments, inside layer 402 or cavity 404 may comprise graphene layers, graphene filling, or other material. In some embodiments, channel 406 may be created within stack 400 in order to allow fluids to pass through stack 400. In some embodiments, channel 406 may comprise a variety of diameters and paths. For example, channel 406 may directly connect both ends of stack 400. In some embodiments, channel 406 may weave through stack 400, as shown in FIG. 4. In some embodiments, channel 406 may be indirect in order to increase the surface area of graphene within stack 400. In this example, a fluid sample passing through stack 400 may come into contact with an increased surface area of the graphene. In some embodiments, channel 406 may weave in such a way that a fluid sample passing through stack 400 may come into contact with multiple faces of sensing units 408-416, as shown in FIG. 4.

FIG. 5 shows a sensing system 500, in accordance with one or more embodiments. In some embodiments, stack 502 may be the same as or similar to stack 400, as shown in FIG. 4. In some embodiments, stack 502 may comprise various sensing units (e.g., sensing unit 506, sensing unit 508, sensing unit 510, sensing unit 512, sensing unit 514, etc.). In some embodiments, stack 502 may comprise any number of sensing units, and different numbers and combinations of sensing units may be used for different applications. For example, sensing units 506-514 may be type C, type C, type A, type F, type A, respectively. In some embodiments, the particular number and combination of sensing units 506-514 may correspond to a particular application, such as sensing air quality in a public area. In some embodiments, stack 502 may include spacers 504. For example, spacers 504 may separate sensing units 506-514. In some embodiments, spacers 504 may electrically isolate the sensing units from each other, for example, such that electric measurements (e.g., resistance) may be made for each individual sensing unit. In some embodiments, spacers 504 may be made of a non-metal or insulating material.

In some embodiments, voltage may be applied to stack 502. In some embodiments, voltage may be applied to stack 502 by a voltage generator 516. For example, sensing units 506-514 may function as resistors. In some embodiments, resistance, voltage drops, or other measurements of each sensing unit may be measured. In some embodiments, spacers 504 may electrically isolate each sensing unit such that resistances of each individual sensing unit may be measured. In some embodiments, the measurements may be converted to digital or electrical signals (e.g., by ADC 518). In some embodiments, processor 520 may receive the digital or electrical signals and may process the signals locally or send the signals to a remote location (e.g., computer system 102) for processing. Techniques for processing the signals are described below in further detail.

In some embodiments, sensing system 500 may further include a battery 522. In some embodiments, sensing system 500 may further include a pump 524 (e.g., air pump, fluid pump, suction pump, or other type of pump). In some embodiments, pump 524 may activate when a request for a measurement is received and may deactivate once the measurement has been taken. In some embodiments, sensing system 500 may additionally include a housing, holder, or cartridge which holds the various components of sensing system 500. For example, a sensing device may comprise the components of sensing system 500 within a housing. In some embodiments, sensing system 500 may be configured in a variety of ways and may include additional or fewer components. In some embodiments, sensing system 500 may form a sensing device or a part of a sensing device.

Returning to FIG. 1, sensing subsystem 120 may include various components shown in FIGS. 3, 4, and 5. For example, sensing subsystem 120 may include sensing units such as sensing unit 300, as shown in FIG. 3. Sensing subsystem 120 may include stack 400 of sensing units, as shown in FIG. 4. Sensing subsystem 120 may include sensing system 500, as shown in FIG. 5. In some embodiments, sensing subsystem 120 may receive a fluid sample. For example, a fluid sample may be an air sample or a liquid sample. In some embodiments, a pump (e.g., pump 524, as shown in FIG. 5) may push or pull the fluid sample through sensing subsystem 120. For example, the fluid sample may be an air sample or other gas sample or a liquid sample. In some embodiments, the fluid sample may be received in response to a request for a test of a fluid. For example, a user of a sensing device (e.g., client device 104) may input a request for fluid testing via user interface subsystem 124.

In some embodiments, sensing subsystem 120 may comprise a communication link to user interface subsystem 124 or to other components of system 100 (e.g., via network 150). In some embodiments, user interface subsystem 124 may be configured to provide an interface between system 100 and the user or other users through which the user or other users may provide information to and receive information from system 100. This enables data, cues, preferences, or instructions and any other communicable items, collectively referred to as “information,” to be communicated between the user and the various components of system 100. In some embodiments, user interface subsystem 124 may be or be included in a computing device, such as a desktop computer, a laptop computer, a smartphone, a tablet computer, a wearable device, an augmented device, or other computing devices. Such computing devices may run one or more electronic applications having graphical user interfaces configured to provide information to or receive information from users. In some embodiments, user interface subsystem 124 may include or communicate with display subsystem 126. For example, one or more test results or other displays may be presented to the user via user interface subsystem 124 or display subsystem 126. It should be noted that although sensing subsystem 120, identification subsystem 122, user interface subsystem 124, and display subsystem 126 are shown in FIG. 1 within a single client device 104a, this is not intended to be limiting. For example, each subsystem may exist together or separately within one or more client device(s) 104.

In some embodiments, the fluid sample may pass through the sensing device (e.g., through stack 502, as shown in FIG. 5). In some embodiments, as the fluid sample passes through the sensing device, the fluid sample may come into contact with various components of the sensing device. For example, the fluid sample may come into contact with the graphene coating or filling of the sensing units. In some embodiments, certain chemicals (e.g., DNA strands) in the fluid sample may bond with the graphene. In some embodiments, the reactions between chemicals in the fluid sample and the graphene of the sensing device may depend on the type of chemical sensitivity of the particular sensing units. For example, the fluid sample may cause different reactions with the sensing units based on the chemical functionality dopants applied to that particular sensing unit. In some embodiments, the sensing units for a particular sensing device may be selected for a particular application. For example, when testing for a particular chemical (e.g., chemical X), sensing units which react with chemical X (e.g., due to the chemical functionality dopants applied to those sensing units) may be selected for the sensing device.

In some embodiments, sensing subsystem 120 may measure resistance of each sensing unit while voltage is applied to the sensing units. For example, an ADC (e.g., ADC 518, as shown in FIG. 5) may measure the resistance of each sensing unit while the fluid sample is passing through the sensing device or after the fluid has passed through the sensing device. In some embodiments, a reaction between chemicals in the fluid sample and the graphene of the sensing device may cause a change to the resistance of a particular sensing unit. For example, a reaction of graphene with a particular chemical may cause structures within the graphene to break down, thereby changing the resistance of the graphene. Therefore, the ADC may detect a particular sensing unit which has reacted with the fluid sample. In some embodiments, the ADC may convert the resistance measurements into digital signals. In some embodiments, information relating to the measurements may be processed at client device 104 or may be sent to computer system 102 for processing. For example, information relating to voltage, changes in resistance, fluid samples, and chemical sensitivities of sensing units which reacted to the fluid samples may be processed by identification subsystem 122 of client device 104 or identification subsystem 110 of computer system 102.

In some embodiments, based on one or more chemical sensitivities of the particular sensing unit, identification subsystem 110 or identification subsystem 122 may identify one or more chemicals in a fluid sample associated with a reaction in the sensing device. For example, if identification subsystem 122 identifies the chemical locally at client device 104, identification subsystem 122 may compare the sensing units which reacted to the fluid sample to a locally stored database. For example, identification subsystem 122 may compare the chemical sensitivities (e.g., based on the chemical functionality dopants applied to the sensing unit), the reaction to the fluid sample (e.g., changes in resistance), and other information about the sensing unit to a locally stored database. The locally stored database may comprise entries having chemical sensitivities, reactions (e.g., changes in resistance), associated chemicals, and other information. For example, identification subsystem 122 may compare chemical sensitivities and a resistance measurement of sensing unit 300, as shown in FIG. 3, to the locally stored database. Identification subsystem 122 may identify a match for the properties and changes of sensing unit 300 in the locally stored database. The database entry may additionally comprise an identification of the chemical or chemicals in the fluid sample which caused the reaction with the sensing unit. Identification subsystem 122 may thereby identify the chemical locally.

In some embodiments, identification subsystem 110 may remotely identify a chemical in a fluid sample associated with a reaction in a sensing device. For example, identification subsystem 110 may receive (e.g., via network 150) chemical sensitivities (e.g., based on the chemical functionality dopants applied to the sensing unit), the reaction to the fluid sample (e.g., change in resistance), and other information about the sensing unit. Identification subsystem 110 may compare the received information to a database (e.g., database 130) comprising entries having chemical sensitivities, reactions (e.g., changes in resistance), associated chemicals, and other information.

In some embodiments, system 100 may utilize both a locally stored database on client device 104 and an external database (e.g., database 130). For example, identification subsystem 122 may first attempt to find a match in the locally stored database and, upon finding no matches, may attempt to find a match in an external database (e.g., database 130). In some embodiments, processing may be done locally (e.g., by identification subsystem 122) when client device 104 lacks connectivity with network 150 (e.g., in remote locations). In some embodiments, a user of client device 104 may specify (e.g., via user interface subsystem 124) desired information to be stored in the locally stored database. For example, if the user plans to test fluid samples for certain chemicals in a remote area with limited connectivity to network 150, the user may download certain database entries or other information to be stored locally on client device 104.

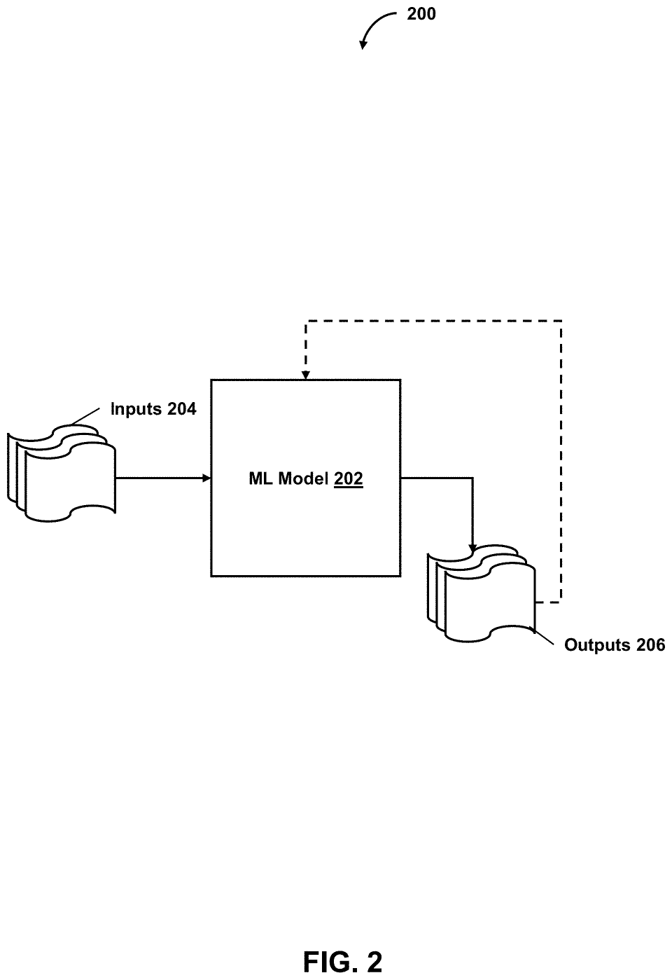

In some embodiments, identification subsystem 110 or identification subsystem 122 may identify a chemical associated with a reaction in the sensing device using a machine learning model. FIG. 2 shows a machine learning model 200 configured to facilitate chemical sensing, in accordance with one or more embodiments. As an example, the machine learning model may include one or more neural networks, although the techniques described in this disclosure are not limited to any particular machine learning model or algorithm. Neural networks may be advantageous in at least certain embodiments because neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it propagates to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

In some embodiments, the prediction model may update its configurations (for example, weights, biases, or other parameters) based on its assessment of the predictions. Database 130 (e.g., as shown in FIG. 1) may include training data and one or more trained prediction models.

As an example, with respect to FIG. 2, machine learning model 202 may take inputs 204 and provide outputs 206. For example, in some embodiments, inputs 204 may comprise training data comprising reactions based on chemical sensitivities (e.g., changes in resistance). In some embodiments, inputs 204 may include labels indicating chemicals associated with the reactions. In this example, outputs 206 may comprise predictions based on the training data. For example, the predictions may comprise predicted chemicals associated with the reactions in the training data. In one use case, outputs 206 may be fed back (for example, active feedback) to machine learning model 202 as input to train machine learning model 202 (e.g., alone or in conjunction with user indications of the accuracy of outputs 206, labels associated with the inputs, or with other reference feedback information). In another use case, machine learning model 202 may update its configurations (e.g., weights, biases, or other parameters) based on its assessment of its prediction (e.g., outputs 206) and reference feedback information (e.g., user indication of accuracy, reference labels, or other information). In another use case, where machine learning model 202 is a neural network, connection weights may be adjusted to reconcile differences between the neural network's prediction and the reference feedback. In a further use case, one or more neurons (or nodes) of the neural network may require that their respective errors are sent backward through the neural network to them to facilitate the update process (e.g., backpropagation of error). Updates to the connection weights may, for example, be reflective of the magnitude of error propagated backward after a forward pass has been completed. In this way, for example, the machine learning model 202 may be trained to generate better predictions.

In some embodiments, machine learning model 200 may be located on client device 104, computer system 102, or another location in network 150. For example, machine learning model 200 may be trained locally on client device 104, remotely on computer system 102, or in both locations. For example, machine learning model 200 may initially be trained remotely using datasets retrieved from database 130. Machine learning model 200 may then be used for fluid tests of the user once it has been trained. In some embodiments, machine learning model 200 may be further trained locally on client device 104 using fluid tests conducted by the user. Machine learning model 200 may thus improve its predictions of associated chemicals as the user conducts fluid tests. In some embodiments, updates to machine learning model 200 may be uploaded to computer system 102 when client device 104 is connected to network 150. In some embodiments, machine learning model 200 may be continuously updated using fluid tests conducted by multiple users with multiple client devices 104a-104n.

In some embodiments, results of a fluid tests may be displayed to the user via display subsystem 126. In some embodiments, display subsystem 126 may be a screen, projector, series of lights, or other display mechanism. In some embodiments, display subsystem 126 may display one or more chemicals identified in the fluid sample (e.g., based on reactions in the sensing device). In some embodiments, display subsystem 126 may display amounts of chemicals identified in the fluid sample. In some embodiments, display subsystem 126 may issue alerts if identified chemicals are hazardous or at a hazardous level. In some embodiments, chemicals detected in a particular ratio (e.g., in a combination or ratios that are hazardous) may cause display subsystem 126 to display an alert. For example, alerts may include lighting up, flashing, displaying a message, or otherwise alerting the user.

In some embodiments, the sensing device (e.g., client device 104) may be single-use or reusable. For example, if the reaction in a sensing unit permanently alters the structure of the sensing unit, the sensing device may not be reusable. If the reactions in the sensing units temporarily alter the structure of the sensing unit or leave the structure of the sensing unit intact, the sensing device may be reusable.

In some embodiments, system 100 may function as a chemical sensing platform. For example, in some embodiments, selectable chemical sensitivities, databases, trained machine learning models, and other components for chemical sensing may be stored remotely (e.g., on computer system 102). Users of client device 104 may select, program, download, or otherwise choose (e.g., via user interface subsystem 124) chemical sensitivities to include in client device 104. In some embodiments, client device 104 may include functionalities other than those described herein for testing fluid samples against chemical sensitivities. For example, client device 104 may be able to identify a chemical fingerprint or signature of a fluid sample using various techniques and may send the chemical fingerprint or signature to computer system 102. Processing of chemical reactions, fingerprints, signatures, or other information may be done remotely at computer system 102 (e.g., using database 130, remote machine learning model 200, etc.). In some embodiments, computer system 102 may send information (e.g., test results) to client device 104 (e.g., for display to the user via display subsystem 126). In some embodiments, a chemical sensing platform of system 100 may be purchased and downloaded to a client device 104. In this example, all training and processing may be done at client device 104. In some embodiments, local training of machine learning model 200 may allow the user to train a model that is specific to the user's data. In some embodiments, a user may download training databases or databases for identifying chemicals from system 100. In some embodiments, a user may create custom databases based on chemicals the user seeks to identify. System 100 may function as a chemical sensing platform using these or other techniques.

Various applications exist for the sensing device and sensing systems described herein. In some embodiments, air samples taken from an individual's breath may identify illnesses or diseases based on the chemical composition of the air samples. For example, a human or pet may breathe on the sensing device of system 100. The air sample may comprise biomarkers associated with particular illnesses or diseases. For example, biomarkers associated with lung cancer include ethanol, isopropanol and acetone. System 100 may detect and identify these or other biomarkers in the breath of an individual. In some embodiments, if the biomarkers are present in certain amounts, ratios, or combinations, system 100 may alert the user of a possible illness or disease. In some embodiments, system 100 may receive an air sample taken from a toilet (e.g., next to the toilet seat). Based on processing the air sample, system 100 may identify pathogens existing in or around the toilet. System 100 may alert a building owner or cleaning staff of the existence of pathogens in or around the toilet. In some embodiments, system 100 may receive a liquid sample such as urine and may process the sample for hormones, pathogens, or other chemicals. For example, system 100 may identify hormone levels which indicate pregnancy. In another example, system 100 may identify chemicals which indicate illness, infection, or disease. In some embodiments, system 100 may identify, at an early stage, conditions requiring medical attention.

In some embodiments, air quality in various environments may be tested by system 100. For example, odor levels in public areas may be tested. For example, a build-up of garbage in public transportation areas may be detected based on odor. In another example, a user in a coal plant or mine may monitor air quality to ensure that toxins have not entered the air. In another example, system 100 may test air samples for oil fumes. System 100 may determine based on the air samples that an oil leak has occurred. System 100 may thereby identify unclean areas or poisonous air quality and may alert a user of the identified chemicals in the air. In some embodiments, system 100 may test air samples near batteries, for example, in electric vehicles. Based on the chemical composition of the air samples, system 100 may identify that a battery is outgassing and may generate an alert of an overcharged, expired, or dead battery, allowing the user to take early action to replace the battery.

In some embodiments, system 100 may receive air samples from food areas (e.g., a kitchen, fridge, preparation counter, etc.). In some embodiments, based on processing the air samples from the food areas, system 100 may identify spoiled or expired food, bacteria, pathogens, or other contaminants in the food area. In some embodiments, system 100 may compare chemical compositions of air samples to previous air samples taken. If system 100 identifies a drastic change in the chemical composition of a new air sample, system 100 may identify that food in the area has spoiled. System 100 may thereby reduce foodborne illnesses. In some embodiments, system 100 may be used to determine shelf life of produce and other products. For example, based on a sample taken from the air surrounding produce, system 100 may identify the chemical levels in the air sample. For example, high levels of ethylene in the air sample may indicate that the produce has a short shelf life due to a high rate of ripening. Additionally, system 100 may identify contaminants of the produce, such as bacteria, funguses, or other contaminants, based on the air sample. System 100 may thus be used to reduce the number of spoiled or contaminated produce that is sold to consumers.

In some embodiments, system 100 may test water quality. For example, system 100 may collect water samples from water bottles, manufacturing plants, water taps, water pipes, water fountains, and other water sources. System 100 may test the water for contaminants at hazardous levels or in hazardous combinations. In some embodiments, any ingestible liquid may be tested for bacteria or other contaminants. For example, juice, soda, alcohol, coffee, or other liquids may be tested to ensure freshness and lack of contaminants.

In some embodiments, system 100 may test air samples received from various outdoor environments. For example, system 100 may test an air sample from crops. Based on the detected in the air sample, system 100 may determine health and other information about the crops. Farmers may test air quality in livestock pens and barns to test for unhealthy conditions. Farmers, or other users may thus use system 100 to monitor the health of livestock, pets, and other animals and environments. Additionally, air samples taken from fields, crops, or lawns may indicate the presence and levels of pesticides, herbicides, and other chemicals. For example, an air sample from a lawn may indicate that chemical levels of the lawn are high. System 100 may therefore alert a user that the lawn is toxic to children or pets in the vicinity.

In some embodiments, for any application described herein, a sensing device having a particular combination of sensing units may be used. For example, to test for contaminants in drinking water, a first set of sensing units may be included in a sensing device. To test air quality in a coal mine, a second set of sensing units may be included in a sensing device. In some embodiments, a ratio of types of sensing units may be important for sensing chemicals in a particular environment. For example, if system 100 is set up to detect twice as much of a first chemical as a second chemical, the sensing device of system 100 may comprise twice as many sensing units having chemical sensitivities for the first chemical as sensing units having chemical sensitivities for the second chemical. In some embodiments, multiple sensing units having the same chemical sensitivity may be included in a sensing device in order to amplify a signal associated with low levels of a corresponding chemical. In some embodiments, sensing devices may comprise various numbers and combinations of sensing units in accordance with the application (i.e., environment and chemicals for which system 100 is testing).

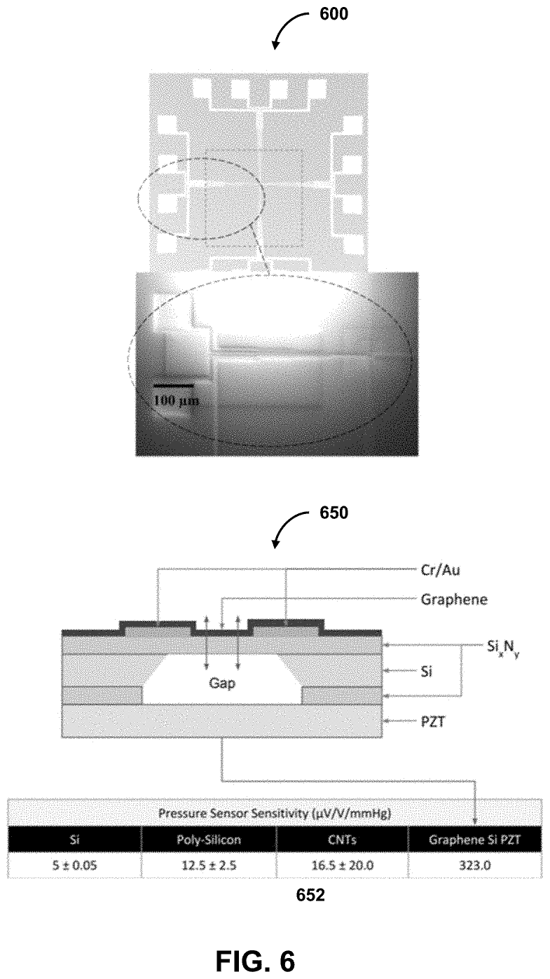

Returning to FIG. 1, system 100 may facilitate sensitive, repeatable, and reliable chemical sensing. System 100 may specifically improve upon repeatability and reliability or prior systems. Background FIG. 6 shows graphene deposited on a silicon test chip 600 on a pressure sensor 650, in accordance with one or more embodiments. As shown in background FIG. 6, various materials may be deposited on silicon test chips on a pressure sensor, and pressure sensor sensitivity (e.g., measured in

$$323 \frac{µV}{V} / mmHg$$

may be different for each material.

According to chart 652, graphene has the highest pressure sensor sensitivity of the materials shown (e.g.

$$323 \frac{µV}{V} / mmHg )$$

and may thus contribute to a chemical sensing system with high sensitivity.

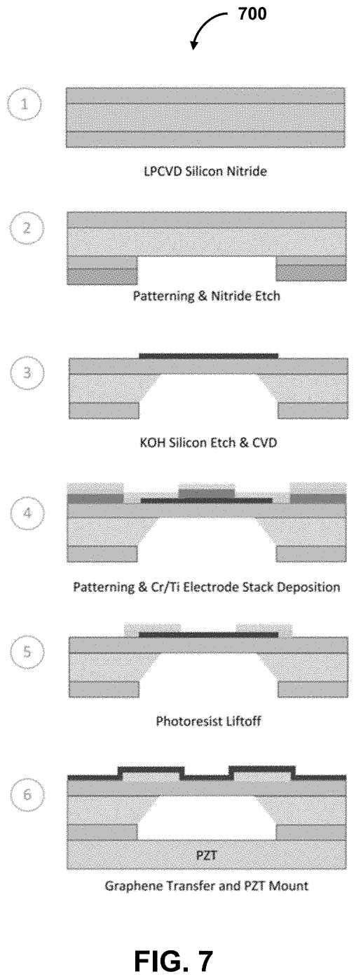

Background FIG. 7 shows a device fabrication process 700, in accordance with one or more embodiments. For example, sensing devices may be processed using various etching, doping, transfer, liftoff, deposition, and other processes. The sensing devices described herein may be manufactured using device fabrication process 700, other processes, or any combination therein.

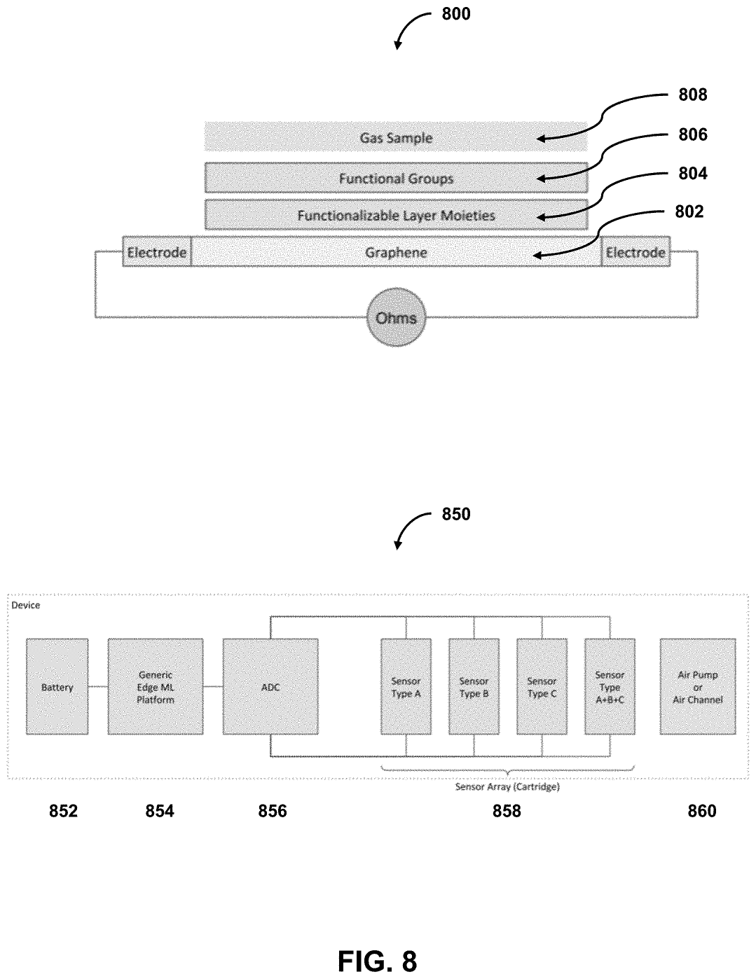

FIG. 8 shows a sensing unit 800 and a sensing device 850, in accordance with one or more embodiments. For example, sensing device 850 may have one or more sensing units 858, each corresponding to one or more chemical sensitivities. The sensing units may have one or more coatings which provide the sensing units with one or more properties. In some embodiments, the sensing units may be combined to create a sensing device 850 having particular chemical sensitivities. In some embodiments, the combination of sensing units 858 in sensing device 850 may correspond to a particular application for which the sensing device is to be used. In some embodiments, system 100 may receive a fluid sample at the sensing device. In some embodiments, the one or more chemical sensitivities may cause one or more sensing units 858 of sensing device 850 to react to a chemical in the fluid sample. In some embodiments, system 100 may identify one or more chemicals associated with the reactions of the sensing units. For example, system 100 may compare the reactions to a database comprising reactions based on chemical sensitivities and chemicals associated with the reactions based on the chemical sensitivities. In some embodiments, system 100 may utilize a machine learning model (e.g., generic edge ML platform 854, as shown in FIG. 8, or ML model 202, as shown in FIG. 2) or a neural network in order to identify the chemicals based on the reaction of the sensing device.

In some embodiments, sensing units 858 may be coated with carbon or an allotrope of carbon, such as graphene. In some embodiments, graphene may be used due to its sensitive properties and ability to bond with chemicals (e.g., smells). Graphene manufacturing processes have allowed CVD graphene to be scalable and integrated with ubiquitous CMOS technology, for example, via growth on deposited copper thin film catalysts on standard silicon/silicon oxygen wafers (e.g., 100-600 mm). Monolayer graphene coverage of over 95% is achieved on 100-600 mm wafer substrates with negligible effects (e.g., confirmed by extensive Raman mappings). Graphene functionalization occurs via attachment at the defect site. CVD processes and the geometric pattern of a graphene layout allow for negligible surface defects and known quantified edges (e.g., perimeter lengths). In some embodiments, functional groups (e.g., described below) attach to the edges of the graphene layout. In some embodiments, a self-selected assembly environment will produce repeatable functional sites and density. This may contribute to a repeatable sensing device.

In some embodiments, graphene may be applied as a layer onto sensing units 858, inserted as a filler into the sensing units, placed within a cavity of the sensing units, or otherwise applied to the sensing units. In some embodiments, the sensing units may additionally be coated with a chemical functionality dopant. For example, the dopant may be an impurity element which is added to the sensing unit in order to alter its properties. In some embodiments, the chemical functionality dopant may determine the type of sensing unit. As shown in FIG. 8, sensing unit 800 may include a graphene layer 802, functionalizable layer moieties 804, and functional groups 806.

As discussed above, the functional group of a particular sensing unit may determine the type of sensing unit, for example, type A, type B, type C, etc. For example, a particular chemical functionality dopant may be applied to sensing unit 800, thereby adding a first chemical functionality sensing unit 800. Sensing unit 800 may thereafter be a first type (e.g., type A) of sensing units. In some embodiments, different chemical functionality dopants may be applied to different series of sensing units such that multiple types of sensing units are manufactured and may be included in a single sensing device (e.g., as shown by sensing units 858). In some embodiments, additional coatings or layers may be applied to the sensing units. For example, dielectric materials, which may insulate the sensing units from electric conduction, may be applied to the sensing units. Metal oxide, DNA dopants, or other layers may be applied to the sensing units to provide the sensing units with various properties. In some embodiments, each coating or layer may be applied using heat (e.g., in a furnace), with pipettes, or using other application techniques.

In some embodiments, sensing device 850 may include a battery 852. In some embodiments, sensing device 850 may include an air pump 860. (e.g., or fluid pump, suction pump, or other type of pump), for example, to pump a gas sample 808 across one or more sensing units. In some embodiments, air pump 860 may pump gas sample 808 across one or more sensing units of a sensing device. In some embodiments, air pump 860 may activate when a request for a measurement is received and may deactivate once the measurement has been taken. In some embodiments, sensing device 850 may include a voltage generator, an analog-to-digital converter (ADC) 856, or some other means by which to apply a voltage to the device.

Returning to FIG. 1, sensing subsystem 120 may include various components shown in FIG. 8. For example, sensing subsystem 120 may include sensing units such as sensing unit 800, as shown in FIG. 8. Sensing subsystem 120 may include sensing device 850, as shown in FIG. 8. In some embodiments, sensing subsystem 120 may comprise a communication link to user interface subsystem 124 or to other components of system 100 (e.g., via network 150). In some embodiments, user interface subsystem 124 may be configured to provide an interface between system 100 and the user or other users through which the user or other users may provide information to and receive information from system 100. This enables data, cues, preferences, or instructions and any other communicable items, collectively referred to as “information,” to be communicated between the user and the various components of system 100.

In some embodiments, user interface subsystem 124 may be or be included in a computing device, such as a desktop computer, a laptop computer, a smartphone, a tablet computer, a wearable device, an augmented device, or other computing devices. Such computing devices may run one or more electronic applications having graphical user interfaces configured to provide information to or receive information from users. In some embodiments, user interface subsystem 124 may include or communicate with display subsystem 126. For example, one or more test results or other displays may be presented to the user via user interface subsystem 124 or display subsystem 126. It should be noted that although sensing subsystem 120, identification subsystem 122, user interface subsystem 124, and display subsystem 126 are shown in FIG. 1 within a single client device 104a, this is not intended to be limiting. For example, each subsystem may exist together or separately within one or more client device(s) 104.

In some embodiments, identification subsystem 110 or identification subsystem 122 may identify a chemical associated with a reaction in the sensing device using a machine learning model. Returning to FIG. 2, machine learning model 200 configured to facilitate sensitive, repeatable, and reliable chemical sensing, in accordance with one or more embodiments. In some embodiments, inputs 204 may comprise training data comprising reactions based on chemical sensitivities (e.g., changes in resistance). In some embodiments, inputs 204 may include labels indicating chemicals associated with the reactions. In this example, outputs 206 may comprise predictions based on the training data. For example, the predictions may comprise predicted chemicals associated with the reactions in the training data. In one use case, outputs 206 may be fed back (for example, active feedback) to machine learning model 202 as input to train machine learning model 202 (e.g., alone or in conjunction with user indications of the accuracy of outputs 206, labels associated with the inputs, or with other reference feedback information). In another use case, machine learning model 202 may update its configurations (e.g., weights, biases, or other parameters) based on its assessment of its prediction (e.g., outputs 206) and reference feedback information (e.g., user indication of accuracy, reference labels, or other information). In another use case, where machine learning model 202 is a neural network, connection weights may be adjusted to reconcile differences between the neural network's prediction and the reference feedback. In a further use case, one or more neurons (or nodes) of the neural network may require that their respective errors are sent backward through the neural network to them to facilitate the update process (e.g., backpropagation of error). Updates to the connection weights may, for example, be reflective of the magnitude of error propagated backward after a forward pass has been completed. In this way, for example, the machine learning model 202 may be trained to generate better predictions.

In some embodiments, system 100 may increase accuracy of the processes described herein by measuring resistivity amplified by applied mechanical strain at a known frequency. Piezoresistivity may describe a change of resistance in a semiconductor due to applied stress. For example, in semiconducting materials (e.g., germanium, polycrystalline silicon, amorphous silicon, silicon carbide, and single crystal silicon), changes in inter-atomic spacing responding from strain affect bandgaps (e.g., energy ranges in a solid where no electronic states can exist). This makes it easier for electrons to be raised into the conduction band of such solids. This results in a change in resistivity of the material. Within a certain range of strain, the relationship between the strain and the change of resistivity is linear. The piezoresistive coefficient, p_{σ}, is therefore defined as:

$${ρ}_{σ} = \frac{( \frac{\partial ρ}{ρ} )}{ɛ} ,$$

where ∂ρ is change in resistivity, p is original resistivity, and £ is strain. The piezoresistive coefficient of semiconducting materials can be several orders of magnitude larger than the geometrical effect of the strain. Semiconductor strain gauges with a very high coefficient of sensitivity can thus be built. Applying this same principle, by measuring changes in resistivity of a layer of functionalized graphene under mechanical strain, a highly sensitive chemical sensor can be built, since the changes in resistivity and rheological properties of the graphene composite are proportional to the number of target molecules bonded to the functional groups in the graphene.

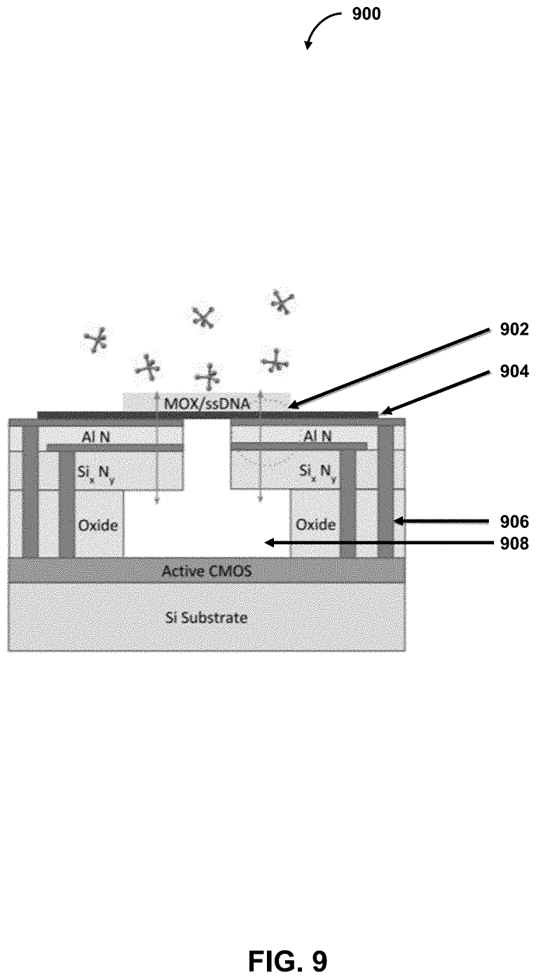

For example, FIG. 9 shows an exposed measurement structure 900, in accordance with one or more embodiments. In some embodiments, exposed measurement structure 900 may include aluminum nitride bimorphs 902, graphene layer 904, one or more vias 906, and other components, as shown in FIG. 9. In some embodiments, various components shown in FIG. 9 may correspond to components shown in FIGS. 4 and 5. In some embodiments, exposed measurement structure 900 may include a cavity 908, which may allow graphene layer 904 to vibrate using aluminum nitride bimorphs 902. In some embodiments, a voltage and a frequency may be applied to exposed measurement structure 900. Resistance may be measured across exposed measurement structure 900 as the applied voltage and frequency are varied. In some embodiments, resistivity may be measured based on variations in the applied voltage and piezoresistivity may be measured based on variations in the applied frequency. The shift in resistance (e.g., measured in ppm) may produce a feature vector. In some embodiments, the feature vector may be used to classify one or more chemicals in a fluid sample.

For example, a fluid sample may pass through or over expose measurement structure 900. In some embodiments, as the fluid sample passes through the sensing device, the fluid sample may come into contact with various components of the sensing device. For example, the fluid sample may come into contact with the graphene coating. In some embodiments, certain chemicals (e.g., DNA strands) in the fluid sample may bond with the graphene. In some embodiments, the reactions between chemicals in the fluid sample and the graphene of the sensing device may depend on the type of chemical sensitivity of the particular sensing units. For example, the fluid sample may cause different reactions with the sensing units based on the chemical functionality dopants applied to that particular sensing unit. In some embodiments, the sensing units for a particular sensing device may be selected for a particular application. For example, when testing for a particular chemical (e.g., chemical X), sensing units which react with chemical X (e.g., due to the chemical functionality dopants applied to those sensing units) may be selected for the sensing device.

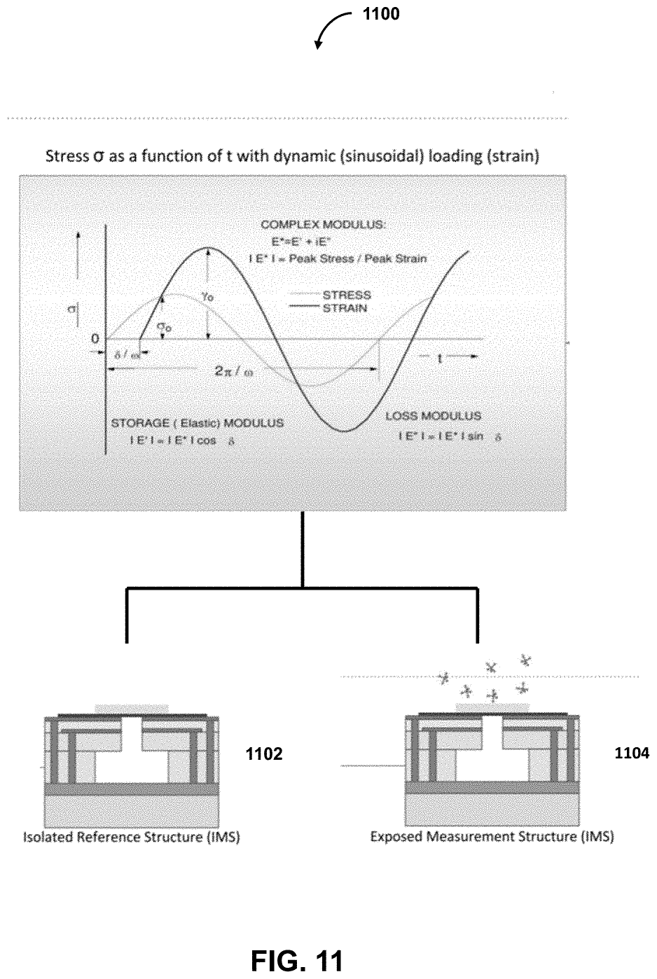

In some embodiments, sensing subsystem 120 may measure resistance of each structure while voltage and frequency are applied to the structures. For example, an ADC (e.g., ADC 856, as shown in FIG. 8) may measure the piezoresistivity and resistivity of each sensing unit while the fluid sample is passing through the sensing device or after the fluid has passed through the sensing device. In some embodiments, a reaction between chemicals in the fluid sample and the graphene layer 904 of the sensing device may cause a change to the piezoresistivity or resistivity of a particular sensing unit. For example, a reaction of graphene with a particular chemical may cause structures within the graphene to break down or may cause molecules within the fluid sample to attach to graphene layer 904, thereby changing the piezoresistivity or resistivity of the graphene. For example, graphene layer 904 may stretch as stress is applied, and the amount that graphene layer 904 stretches may depend on the frequency at which the stress is applied or the resistance of the graphene layer 904 (e.g., due to the stretching of the graphene layer or due to molecules attaching to the graphene layer). Stress may be applied at various frequencies by a piezoelectric plate in order to allow the sensing system to detect chemicals that are accessible at those frequencies (e.g., as discussed in greater detail in relation to FIG. 11). The ADC may detect a particular sensing unit which has reacted with the fluid sample. In some embodiments, the ADC may convert resistance measurements into digital signals. In some embodiments, information relating to the measurements may be processed at client device 104 or may be sent to computer system 102 for processing. For example, information relating to voltage, frequency, changes in piezoresistivity or resistivity, fluid samples, and chemical sensitivities of sensing units which reacted to the fluid samples may be processed by identification subsystem 122 of client device 104 or identification subsystem 110 of computer system 102.

In some embodiments, based on one or more chemical sensitivities of the particular sensing unit, identification subsystem 110 or identification subsystem 122 may identify one or more chemicals in a fluid sample associated with a reaction in the sensing device. For example, if identification subsystem 122 identifies the chemical locally at client device 104, identification subsystem 122 may compare the sensing units which reacted to the fluid sample to a remote or locally-stored database. For example, identification subsystem 122 may compare the chemical sensitivities (e.g., based on the chemical functionality dopants applied to the sensing unit), the reaction to the fluid sample (e.g., changes in resistance), and other information about the sensing unit to a remote or locally-stored database. The databases may comprise entries having chemical sensitivities, reactions (e.g., changes in resistance), associated chemicals, and other information. For example, identification subsystem 122 may compare chemical sensitivities and a resistance measurement of sensing unit 800, as shown in FIG. 8, to the one or more databases. Identification subsystem 122 may identify a match for the properties and changes of in piezoresistivity and resistivity in one or more database. The database entry may additionally comprise an identification of the chemical or chemicals in the fluid sample which caused the reaction with the sensing unit. Identification subsystem 122 may thereby identify the chemical in the fluid sample.

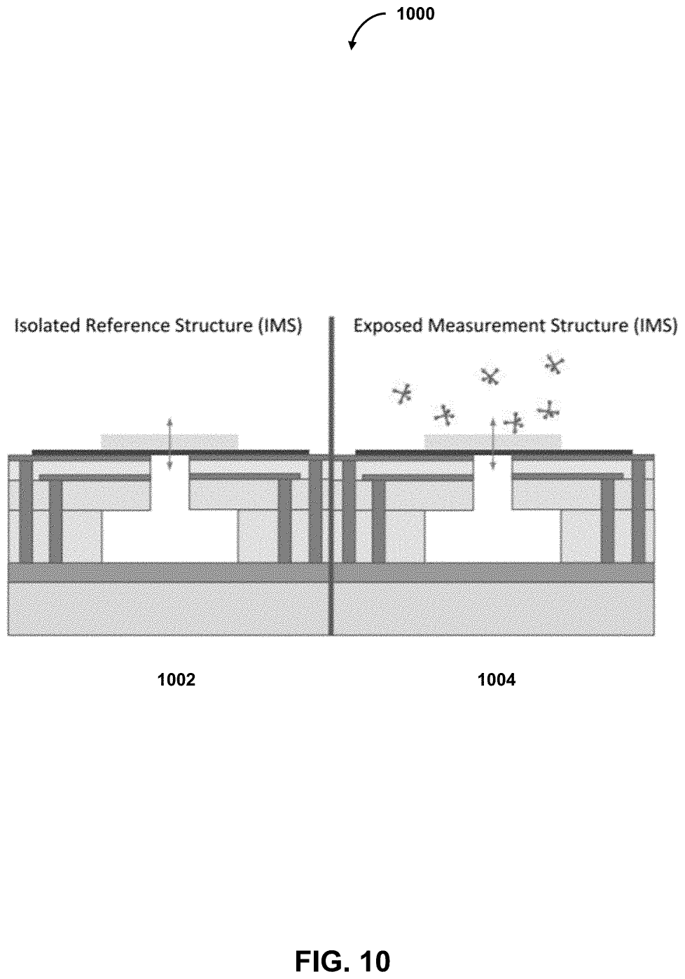

FIG. 10 shows simultaneous excitation 1000 of an isolated reference structure 1002 and an exposed measurement structure 1004, in accordance with one or more embodiments. In some embodiments, system 100 may improve repeatability and manufacturing variability of the processes described herein by simultaneously exiting an isolated reference structure (e.g., isolated reference structure 1002) and an exposed measurement structure (e.g., exposed measurement structure 1004). In some embodiments, exposed measurement structure 1004 may correspond to exposed measurement structure 900, as shown in FIG. 9.

FIG. 11 shows a stress function 1100 with an isolated reference structure 1102 and an exposed measurement structure 1104, in accordance with one or more embodiments. In some embodiments, isolated reference structure 1102 may correspond to isolated reference structure 1002, as shown in FIG. 10, and exposed measurement structure 1104 may correspond to exposed measurement structure 900, as shown in FIG. 9, or exposed measurement structure 1004, as shown in FIG. 10. Stress function 1100 shows stress as a function of time with dynamic (e.g., sinusoidal) applied strain. In some embodiments, the stress function reflects the frequency response from both isolated reference structure 1102 and an exposed measurement structure 1104, which are excited simultaneously. In some embodiments, a modulus of graphene and composite graphene (e.g., graphene with functional groups) may be extracted from the frequency response. The change in moduli are proportional to the attachments or coatings or dopants on the graphene. This modulus may be used to create a repeatable structure, for example, by verifying functional density and by matching quantified functional density to a specific sensor response. In some embodiments, this may improve repeatability and manufacturing variability of the processes described herein. The modulus can also be used as an in-process quality measurement to ensure a repeatable composite graphene structure and to validate the change in resistivity measurements. Furthermore, the resonant frequencies are known to raise the temperature of the graphene, which can be used for refreshing the graphene to shed the detected species attached to the functional groups on the graphene and make the site available for the next attachment and or detection.

In some embodiments, FIG. 11 illustrates stress applied and a resultant strain that amplifies resistivity. An ability to apply a range of stresses and oscillate at various frequencies may allow the sensing system to detect chemicals that are accessible at those frequencies. Additionally, given the ability to oscillate at various frequencies, the sensing system may be able to regenerate a sensor after measurements have been taken (e.g., heat-induced removal of the surface attachments or mechanical vibration-induced removal). In addition to the resistivity changes, the applied stress and strain response of the functional groups on the graphene surface (e.g., due to their mass) may also provide a way to detect species on the surface.

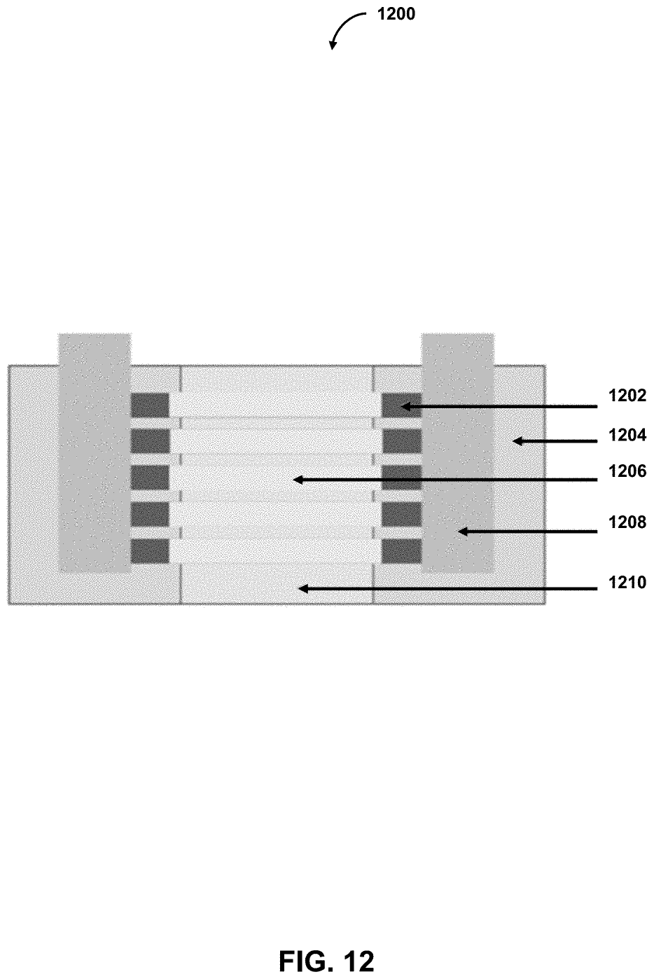

FIG. 12 shows a plane view of a sensor 1200, in accordance with one or more embodiments. In some embodiments, sensor 1200 may correspond to exposed measurement structure 900, as shown in FIG. 9. In some embodiments, sensor 1200 may include graphene 1202, aluminum nitride bimorph 1204, functionalized ssDNA/metal oxide 1206, CR/Au electrodes 1208, cavity 1210, and any additional components. In some embodiments, the structure of sensor 1200, as shown in FIG. 12, maximizes the predictable formation of graphene defects. For example, this structure has an increased number of edges with a known perimeter, which creates probable sites for functional groups to attach repeatedly and reliably and enables control over a number of functional groups that can be attached (e.g., functional density). To an extent this preserves the graphene surface from functionalization, preserving the quality of electrical conduction over the surface and thereby allowing sensor 1200 to obtain additional feature vectors for classifying chemicals.

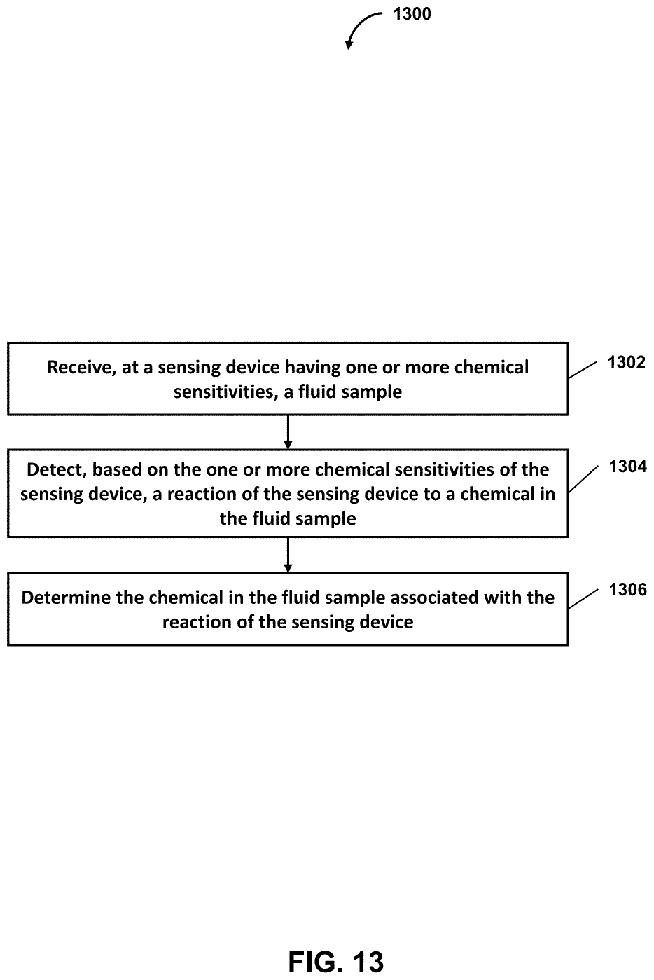

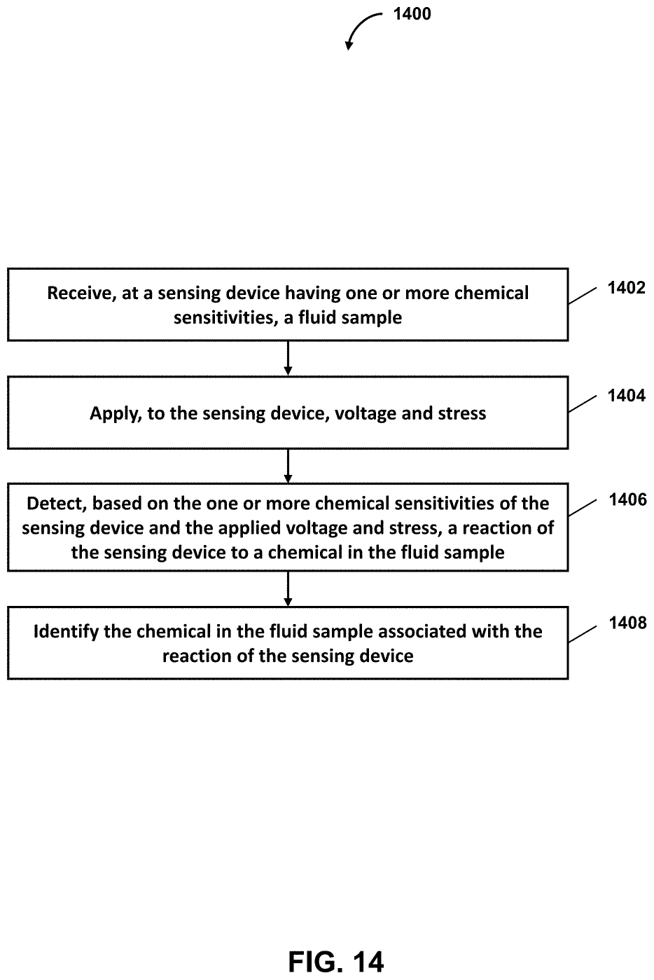

FIGS. 13 and 14 are example flowcharts of processing operations of methods that enable the various features and functionality of the system as described in detail above. The processing operations of each method presented below are intended to be illustrative and non-limiting. In some embodiments, for example, the methods may be accomplished with one or more additional operations not described, and/or without one or more of the operations discussed. Additionally, the order in which the processing operations of the methods are illustrated (and described below) is not intended to be limiting.

In some embodiments, the methods may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on an electronic storage medium. The processing devices may include one or more devices configured through hardware, firmware, and/or software to be specifically designed for execution of one or more of the operations of the methods.

FIG. 13 shows a flowchart 1300 of a method of facilitating chemical sensing, in accordance with one or more embodiments. In an operation 1302, a fluid sample may be received at a sensing device having one or more chemical sensitivities. In some embodiments, the fluid sample may be gaseous or liquid. Operation 1302 may be performed by a subsystem that is the same as or similar to sensing subsystem 120.

In an operation 1304, a reaction of the sensing device to a chemical in the fluid sample may be detected. In some embodiments, the reaction may be based on the one or more chemical sensitivities of the sensing device. Operation 1304 may be performed by a subsystem that is the same as or similar to sensing subsystem 120. In an operation 1306, the chemical in the fluid sample associated with the reaction of the sensing device may be determined. Operation 1306 may be performed by a subsystem that is the same as or similar to identification subsystem 110 or identification subsystem 122.

FIG. 14 shows a flowchart 1400 of a method of facilitating chemical sensing, in accordance with one or more embodiments. In an operation 1402, a fluid sample may be received at a sensing device having one or more chemical sensitivities. Operation 1402 may be performed by a subsystem that is the same as or similar to sensing subsystem 120. In an operation 1404, voltage and stress may be applied to the sensing device. Operation 1404 may be performed by a subsystem that is the same as or similar to sensing subsystem 120.

In an operation 1406, a reaction of the sensing device to a chemical in the fluid sample may be detected. In some embodiments, the reaction may be detected based on the one or more chemical sensitivities and the applied voltage and stress. Operation 1406 may be performed by a subsystem that is the same as or similar to sensing subsystem 120. In an operation 1408, the chemical in the fluid sample associated with the reaction of the sensing device may be identified. Operation 1408 may be performed by a subsystem that is the same as or similar to identification subsystem 110 or identification subsystem 122.

In some embodiments, the various computers and subsystems illustrated in FIG. 1 may include one or more computing devices that are programmed to perform the functions described herein. The computing devices may include one or more electronic storages (e.g., database(s) 130 or other electronic storages), one or more physical processors programmed with one or more computer program instructions, and/or other components. The computing devices may include communication lines or ports to enable the exchange of information within a network (e.g., network 150) or other computing platforms via wired or wireless techniques (e.g., Ethernet, fiber optics, coaxial cable, Wi-Fi, Bluetooth, near field communication, or other technologies). The computing devices may include a plurality of hardware, software, and/or firmware components operating together. For example, the computing devices may be implemented by a cloud of computing platforms operating together as the computing devices.

The electronic storages may include non-transitory storage media that electronically stores information. The storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical-charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

The processors may be programmed to provide information processing capabilities in the computing devices. As such, the processors may include one or more of digital processors, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 120-126, subsystem 110, and/or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 120-126 and subsystem 110 described herein is for illustrative purposes, and is not intended to be limiting, as any of subsystems 120-126 or subsystem 110 may provide more or less functionality than is described. For example, one or more of subsystems 120-126 or subsystem 110 may be eliminated, and some or all of its functionality may be provided by other ones of subsystems 120-126 or subsystem 110. As another example, additional subsystems may be programmed to perform some or all of the functionality attributed herein to one of subsystems 120-126 or subsystem 110.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The present techniques will be better understood with reference to the following enumerated embodiments:

1. A sensing device, comprising: a series of sensing units, wherein each of the sensing units comprises: a base layer; a first coating on the base layer; a second coating on the base layer; a third coating on the base layer; a series of spacers between the sensing units of the series of sensing units; and a housing.
2. The sensing device of embodiment 1, wherein the base layer is a metal tube.
3. The sensing device of any of the preceding embodiments, wherein the first coating comprises a graphene coating.
4. The sensing device of any of the preceding embodiments, wherein the second coating comprises a chemical functionality dopant.
5. The sensing device of any of the preceding embodiments, wherein the second coating corresponds to a chemical sensitivity of the sensing device.
6. The sensing device of any of the preceding embodiments, wherein the third coating comprises a metal oxide.
7. The sensing device of any of the preceding embodiments, wherein the third coating comprises a DNA dopant.
8. The sensing device of any of the preceding embodiments, further comprising: a voltage generator configured to generate a voltage across the series of sensing units; an analog-to-digital converter configured to convert resistances across each of the series of sensing units to electrical signals; and a processor configured to process the electrical signals.
9. The sensing device of any of the preceding embodiments, further comprising a battery.
10. The sensing device of any of the preceding embodiments, further comprising a channel through which fluids are able to pass.
11. A system for sensing chemicals, the system comprising: a computer system that comprises one or more processors programmed with computer program instructions that, when executed, cause the computer system to: receive, at a sensing device having one or more chemical sensitivities, a fluid sample; detect, based on the one or more chemical sensitivities of the sensing device, a reaction of the sensing device to a chemical in the fluid sample; and identify the chemical in the fluid sample associated with the reaction of the sensing device.
12. The system of embodiment 11, wherein the computer system is further caused to: provide a reaction based on a chemical sensitivity as input to a neural network to cause the neural network to generate a predicted associated chemical; obtain feedback indicating an associated chemical; and provide the feedback as reference feedback to the neural network to cause the neural network to assess the feedback against the predicted associated chemical, the neural network being updated based on the assessment of the feedback.
13. The system of embodiment 12, wherein the chemical in the fluid sample associated with the reaction of the sensing device is identified using the updated neural network.
14. The system of embodiment 12, wherein the computer system is further caused to: retrieve a neural network, wherein the neural network is trained to predict chemicals associated with reactions of sensing devices based on chemical sensitivities; and provide a reaction based on a chemical sensitivity as input to the neural network to cause the neural network to generate a predicted associated chemical.
15. The system of any of the preceding embodiments, wherein to identify the chemical in the fluid sample associated with the reaction of the sensing device, the computer system is further caused to: compare the reaction to a database comprising reactions based on chemical sensitivities and corresponding chemicals; and identify a matching reaction based on chemical sensitivities and a corresponding chemical.
16. The system of any of the preceding embodiments, wherein the reaction comprises a resistance change associated with a chemical sensitivity of the one or more chemical sensitivities.
17. The system of any of the preceding embodiments, wherein the fluid sample is liquid or gaseous.
18. A system for sensing chemicals, the system comprising: a computer system that comprises one or more processors programmed with computer program instructions that, when executed, cause the computer system to: receive, at a sensing device having one or more chemical sensitivities, a fluid sample; apply, to the sensing device, stress; detect, based on the one or more chemical sensitivities of the sensing device and the applied stress, a reaction of the sensing device to a chemical in the fluid sample; and identify the chemical in the fluid sample associated with the reaction of the sensing device.
19. The system of embodiment 18, wherein the computer system is further caused to apply, to the sensing device, motion.
20. The system of embodiment 19, wherein the motion is applied at one or more resonance frequencies.
21. The system of embodiment 20, wherein the reaction comprises a change in resistivity or piezoresistivity amplified by the applied stress and motion and the one or more resonance frequencies associated with a chemical sensitivity of the one or more chemical sensitivities.
22. The system of embodiment 20, wherein the reaction comprises a change in the one or more resonance frequencies.
23. The system of any of the preceding embodiments, wherein the computer system is further caused to: provide a reaction based on a chemical sensitivity and applied stress as input to a neural network to cause the neural network to generate a predicted associated chemical; obtain feedback indicating an associated chemical; and provide the feedback as reference feedback to the neural network to cause the neural network to assess the feedback against the predicted associated chemical, the neural network being updated based on the assessment of the feedback.
24. The system of embodiment 23, wherein the chemical in the fluid sample associated with the reaction of the sensing device is identified using the updated neural network.
25. A method being implemented by one or more processors executing computer program instructions that, when executed, perform the method comprising any of embodiments 1-24.
26. A tangible, non-transitory, machine-readable medium storing instructions that, when executed by a data processing apparatus, causes the data processing apparatus to perform operations comprising those of any of embodiments 1-24.

What is claimed is:

1. A sensing device, comprising:
a series of sensing units, wherein each of the sensing units comprises:
a base layer;
a first coating on the base layer;
a second coating on the base layer;
a third coating on the base layer;
a series of spacers between the sensing units of the series of sensing units; and
a housing.

2. The sensing device of claim 1, wherein the base layer is a metal tube.

3. The sensing device of claim 1, wherein the first coating comprises a graphene coating.

4. The sensing device of claim 1, wherein the second coating comprises a chemical functionality dopant.

5. The sensing device of claim 1, wherein the second coating corresponds to a chemical sensitivity of the sensing device.

6. The sensing device of claim 1, wherein the third coating comprises a metal oxide.

7. The sensing device of claim 1, wherein the third coating comprises a DNA dopant.

8. The sensing device of claim 1, further comprising:
a voltage generator configured to generate a voltage across the series of sensing units;
an analog-to-digital converter configured to convert resistances across each of the series of sensing units to electrical signals; and
a processor configured to process the electrical signals.

9. The sensing device of claim 1, further comprising a battery.

10. The sensing device of claim 1, further comprising a channel through which fluids are able to pass.

11. A system for sensing chemicals, the system comprising:
a computer system that comprises one or more processors programmed with computer program instructions that, when executed, cause the computer system to:
receive, at a sensing device having one or more chemical sensitivities, a fluid sample;
detect, based on the one or more chemical sensitivities of the sensing device, a reaction of the sensing device to a chemical in the fluid sample; and
identify the chemical in the fluid sample associated with the reaction of the sensing device.

12. The system of claim 11, wherein the computer system is further caused to:
provide a reaction based on a chemical sensitivity as input to a neural network to cause the neural network to generate a predicted associated chemical;
obtain feedback indicating an associated chemical; and
provide the feedback as reference feedback to the neural network to cause the neural network to assess the feedback against the predicted associated chemical, the neural network being updated based on the assessment of the feedback.

13. The system of claim 12, wherein the chemical in the fluid sample associated with the reaction of the sensing device is identified using the updated neural network.

14. The system of claim 11, wherein the computer system is further caused to:
retrieve a neural network, wherein the neural network is trained to predict chemicals associated with reactions of sensing devices based on chemical sensitivities; and
provide a reaction based on a chemical sensitivity as input to the neural network to cause the neural network to generate a predicted associated chemical.

15. The system of claim 11, wherein to identify the chemical in the fluid sample associated with the reaction of the sensing device, the computer system is further caused to:
compare the reaction to a database comprising reactions based on chemical sensitivities and corresponding chemicals; and
identify a matching reaction based on chemical sensitivities and a corresponding chemical.

16. The system of claim 11, wherein the reaction comprises a resistance change associated with a chemical sensitivity of the one or more chemical sensitivities.

17. The system of claim 11, wherein the fluid sample is liquid or gaseous.

18. A system for sensing chemicals, the system comprising:
a computer system that comprises one or more processors programmed with computer program instructions that, when executed, cause the computer system to:
receive, at a sensing device having one or more chemical sensitivities, a fluid sample;
apply, to the sensing device, stress;
detect, based on the one or more chemical sensitivities of the sensing device and the applied stress, a reaction of the sensing device to a chemical in the fluid sample; and
identify the chemical in the fluid sample associated with the reaction of the sensing device.

19. The system of claim 18, wherein the computer system is further caused to apply, to the sensing device, motion.

20. The system of claim 19, wherein the motion is applied at one or more resonance frequencies.

21. The system of claim 20, wherein the reaction comprises a change in resistivity or piezoresistivity amplified by the applied stress and motion and the one or more resonance frequencies associated with a chemical sensitivity of the one or more chemical sensitivities.

22. The system of claim 20, wherein the reaction comprises a change in the one or more resonance frequencies.

23. The system of claim 18, wherein the computer system is further caused to:
provide a reaction based on a chemical sensitivity and applied stress as input to a neural network to cause the neural network to generate a predicted associated chemical;
obtain feedback indicating an associated chemical; and
provide the feedback as reference feedback to the neural network to cause the neural network to assess the feedback against the predicted associated chemical, the neural network being updated based on the assessment of the feedback.

24. The system of claim 23, wherein the chemical in the fluid sample associated with the reaction of the sensing device is identified using the updated neural network.
